(19) United States
(12) Patent Application Publication
(10) Pub. No.: US 2008/0122344 A1
(43) Pub. Date: May 29, 2008

(54) ORGANIC LIGHT EMITTING COMPOUND AND ORGANIC LIGHT EMITTING DEVICE COMPRISING THE SAME, AND METHOD OF MANUFACTURING THE ORGANIC LIGHT EMITTING DEVICE

(75) Inventors: Dong-woo SHIN, Yongin-si (KR); Eun-sil HAN, Yongin-si (KR); Woon-jung PAEK, Yongin-si (KR); Yi-yeol LYU, Yongin-si (KR); O-hyun KWON, Yongin-si (KR); Myeong-suk KIM, Yongin-si (KR); Byoung-ki CHOI, Yongin-si (KR); Jhun-mo SON, Yongin-si (KR); Young-mok SON, Yongin-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 11/756,105

(22) Filed: May 31, 2007

(30) Foreign Application Priority Data
Nov. 24, 2006 (KR) 10-2006-0117251

(51) Int. Cl. H01J 1/63; C07D 209/56; H01J 9/00

(52) U.S. Cl. 313/504; 445/24; 548/418; 548/420

(57) ABSTRACT

Provided are an organic light emitting compound represented by Formula 1 below, an organic light emitting device comprising the same, and a method of manufacturing the light emitting device:

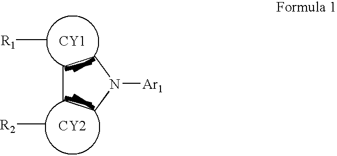

wherein CY1, CY2, Ar₁, R₁ and R₂ are described in the detailed description of the invention. An organic light emitting device comprising the organic light emitting compound has low turn-on voltage, high efficiency, high color purity and high luminance.

### CROSS-REFERENCE TO RELATED PATENT APPLICATION

This application claims priority to Korean Patent Application No. 10-2006-0117251, filed on Nov. 24, 2006, and all the benefits accruing therefrom under 35 U.S.C. §119, the contents of which are incorporated herein by reference in their entirety.

### BACKGROUND OF THE INVENTION

1. Field of the Invention

The present invention relates to an organic light emitting compound, an organic light emitting device comprising the same, and a method of manufacturing the organic light emitting device, and more specifically, to an organic light emitting compound that has good electrical properties, high thermal stability and high photochemical stability, and that has a low turn-on voltage, a high efficiency, a high color purity and a high luminance when used in an organic light emitting device.

2. Description of the Related Art

Light emitting devices (LED or LEDs) have wide viewing angles, excellent contrast, and quick response time. LEDs can be categorized into inorganic LEDs and organic LEDs (OLED or OLEDs) according to the material used to form the emission layer of the LEDs. Because OLEDs are brighter, have a lower operating voltage and quicker response time compared to inorganic LEDs, and can realize multi color images, extensive research has been conducted into OLEDs.

In general, an OLED has an anode/organic emission layer/cathode structure. An OLED can also have various other structures, such as an anode/hole injection layer/hole transport layer/emission layer/electron transport layer/electron injection layer/cathode structure or an anode/hole injection layer/hole transport layer/emission layer/hole blocking layer/electron transport layer/electron injection layer/cathode structure.

Materials used in OLEDs can be classified into vacuum deposition materials and solution coating materials depending on the method of preparing the organic layer of the OLEDs. The vacuum deposition material can have a vapor pressure of 10⁻⁶ torr (1.33×10⁻⁷ kilopascals (kPa)) or more, at 500° C. or less, and preferably may be a low molecular weight material having a molecular weight of 1,200 atomic mass units (amu) or less. The solution coating material should have high solubility with respect to a solvent so that it can be prepared as a solution, and comprises an aromatic group or a heterocyclic ring.

When OLEDs are manufactured using vacuum deposition, the manufacturing costs increase because of the use of a vacuum system. In addition, when a shadow mask method is used for preparing pixels for natural color display, it is difficult to prepare high resolution pixels. On the other hand, when OLEDs are manufactured using a solution coating method such as inkjet printing, screen printing, spin coating, or the like, the manufacturing method is simple and inexpensive, and the OLEDs can have relatively good resolution compared with OLEDs manufactured using the shadow mask method.

However, in the case of molecules emitting blue light, materials that can be used in solution coating are inferior to materials that can be used in vacuum deposition in terms of thermal stability, color purity and the like. In addition, although such materials having good performance are used for preparing the organic layer, the material is gradually crystallized after the organic layer is prepared so that the size of the crystal corresponds to the wavelength range of visible light. As a result, visible light is diffused so that a whitening phenomenon can occur, and pin holes, or the like, can be formed, thus easily causing degradation of the device.

Japanese Patent Publication No. 1999-003782 discloses anthracene substituted with two naphthyl groups as a compound that can be used in an emission layer or a hole injection layer. However, the compound does not have satisfactorily good solubility with respect to a solvent, and OLEDs using such compound do not produce satisfactory characteristics.

Therefore, there is a demand for a compound that can form an organic layer having excellent properties that can be used in an OLED. Accordingly, there is still need for development of an OLED that has an improved turn-on voltage, a high luminance, a high efficiency and a high color purity using a blue-light emitting compound that has high thermal stability and can form an organic layer having excellent properties.

### SUMMARY OF THE INVENTION

Provided herein is an organic light emitting compound having a good solubility, a high color purity and a high thermal stability.

Also provided herein is an OLED comprising an organic layer formed of the organic light emitting compound, which has an improved turn-on voltage, an improved efficiency, color purity and luminance.

Also provided herein is a method of manufacturing an OLED using the organic light emitting compound.

According to an embodiment, there is provided an organic light emitting compound represented by Formula 1 below:

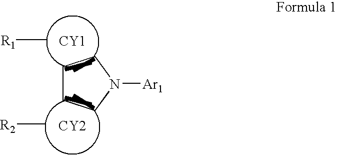

where CY1 and CY2 are each independently a benzene ring or a naphthalene ring; Ar₁ is a substituted or unsubstituted C₃₁-C₁₀₀ aryl group; R₁ and R₂ are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group; and with the proviso that CY1 and CY2 are not both benzene rings.

According to another embodiment, there is provided an OLED comprising: a first electrode; a second electrode; and an organic layer interposed between the first electrode and the second electrode, wherein the organic layer comprises the organic light emitting compound described above.

According to another embodiment, there is provided a method of manufacturing an OLED comprising: forming a first electrode; forming an organic thin film by disposing the organic light emitting compound described above on the first electrode; and forming a second electrode on the organic thin film.

### BRIEF DESCRIPTION OF THE DRAWINGS

The above and other features and advantages will become more apparent by describing in detail exemplary embodiments thereof with reference to the attached drawings in which:

FIGS. 1A through 1C are schematic cross-sectional views illustrating exemplary structures of an;

FIG. 2 is a graph showing experimental results of ultraviolet (UV) and photoluminescence (PL) spectra of Compound 4, which is an organic light emitting compound; and

FIG. 3 is a graph showing the electrical efficiency curve of Sample 1, which is an OLED manufactured Compound 4 according to an embodiment described herein.

### DETAILED DESCRIPTION OF THE INVENTION

The invention is described more fully hereinafter with reference to the accompanying drawings, in which embodiments of the invention are shown. This invention, however, should not be construed as limited to the embodiments set forth herein. Rather, these embodiments are provided to convey the scope of the invention to those skilled in the art. In the drawings, the size and relative sizes of elements and regions may be exaggerated for clarity.

It will be understood that when an element or layer is referred to as being “on,” “interposed,” “disposed,” or “between” another element or layer, it can be directly on, interposed, disposed, or between the other element or layer or intervening elements or layers may be present.

It will be understood that, although the terms first, second, third, and the like may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, first element, component, region, layer or section discussed below could be termed second element, component, region, layer or section without departing from the teachings of the present invention.

As used herein, the singular forms “a,” “an” and “the” are intended to comprise the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

An organic light emitting compound according to an embodiment is represented by Formula 1 below:

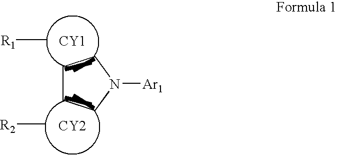

where CY1 and CY2 are each independently a benzene ring or a naphthalene ring; Ar₁ is a substituted or unsubstituted C₃₁-C₁₀₀ aryl group; R₁ and R₂ are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group; with the proviso that CY1 and CY2 are not both benzene rings.

In addition, an organic light emitting compound according to another embodiment can be represented by Formula 2 below:

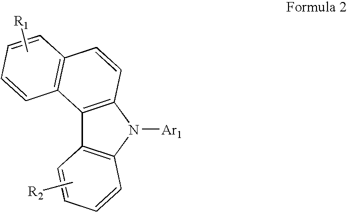

where Ar₁ is a substituted or unsubstituted C₃₁-C₁₀₀ aryl group; R₁ and R₂ are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group.

In addition, an organic light emitting compound according to another embodiment can be represented by Formula 3 below:

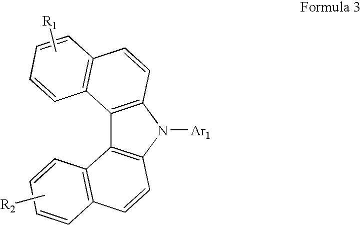

where Ar₁ is a substituted or unsubstituted C₃₁-C₁₀₀ aryl group; R₁ and R₂ are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group.

The aryl group is a monovalent group having an aromatic ring system, and can comprise at least two ring systems. The at least two ring systems may be attached or fused together. The heteroaryl group is a group in which at least one carbon atom of the aryl group is substituted with at least one selected from the group consisting of nitrogen (N), oxygen (O), sulfur (S) and phosphorus (P). Meanwhile, the cycloalkyl group refers to an alkyl group having a ring system, and the heterocycloalkyl group is a group in which at least one carbon atom of the cycloalkyl group is substituted with at least one atom selected from the group consisting of N, O, S and P. Of Formulae 1 through 3, carbazole derivatives and the aryl group bound thereto increase thermal stability and photochemical stability of the organic light emitting compounds represented by Formulae 1 through 3. In addition, R₁ and R₂ increase solubility and the amorphous property of the organic light emitting compounds represented by Formulae 1 through 3 so as to improve film processability. The organic light emitting compounds represented by Formulae 1 through 3 are materials suitable for forming an organic layer of an OLED, wherein the organic layer is interposed between a first electrode and a second electrode. The organic light emitting compounds represented by Formulae 1 through 3 are suitable for an organic layer of an OLED, specifically, an emission layer, a hole injection layer or a hole transport layer, and can also be used as a dopant material in addition to a host material.

Substituents of the alkyl group, the alkoxy group, the aryl group, the heteroaryl group, the cycloalkyl group and the heterocycloalkyl group may selected from the group consisting of —F; —Cl; —Br; —CN; —NO₂; —OH; an unsubstituted C₁-C₅₀ alkyl group or a C₁-C₅₀ alkyl group substituted with —F, —Cl, —Br, —CN, —NO₂ or —OH; an unsubstituted C₁-C₅₀ alkoxy group or a C₁-C₅₀ alkoxy group substituted with —F, —Cl, —Br, —CN, —NO₂ or —OH; an unsubstituted C₆-C₅₀ aryl group or a C₆-C₅₀ aryl group substituted with a C₁-C₅₀ alkyl group, a C₁-C₅₀ alkoxy group, —F, —Cl, —Br, —CN, —NO₂ or —OH; an unsubstituted C₂-C₅₀ heteroaryl group or a C₂-C₅₀ heteroaryl group substituted with a C₁-C₅₀ alkyl group, a C₁-C₅₀ alkoxy group, —F, —Cl, —Br, —CN, —NO₂ or —OH; an unsubstituted C₅-C₅₀ cycloalkyl group or a C₅-C₅₀ cycloalkyl group substituted with a C₁-C₅₀ alkyl group, a C₁-C₅₀ alkoxy group, —F, —Cl, —Br, —CN, —NO₂ or —OH; an unsubstituted C₅-C₅₀ heterocycloalkyl group or a C₅-C₅₀ heterocycloalkyl group substituted with a C₁-C₅₀ alkyl group, a C₁-C₅₀ alkoxy group, —F, —Cl, —Br, —CN, —NO₂ or —OH; and —N(Z₉)(Z₁₀) where Z₉ and Z₁₀ may be each independently a hydrogen atom, a C₁-C₅₀ alkyl group, or a C₆-C₅₀ aryl group substituted with a C₁-C₅₀ alkyl group.

More specifically, Ar₁ is selected from the group consisting of a di(C₆-C₅₀ aryl)benzo[k]fluoranthenyl group, a (C₁₁-C₅₀ aryl)benzo[k]fluoranthenyl group, a (pentaC₆-C₅₀ aryl)phenyl group, a (C₁₃-C₅₀ aryl)terphenyl group, a 9,9-di(C₁₂-C₅₀aryl)fluorenyl group, a (C₁₅-C₅₀ aryl)fluoranthenyl group, a 9-(C₆-C₅₀ aryl)-10-(C₁₁-C₅₀aryl)anthryl group, a (C₁₃-C₅₀ aryl)naphthacenyl group, a (C₁₇-C₅₀ aryl)anthryl group, a ((C₁₁-C₅₀ aryl)naphthyl)naphthyl group, a (C₆-C₅₀ aryl)sexyphenyl group, a ter(di(C₆-C₅₀ aryl)fluorenyl group, a bis(biphenylene)-9,9-di(C₆-C₅₀ aryl)fluorenyl group, a (C₁₁-C₅₀ aryl)benzo[j]fluoranthenyl group, a (C₂₃-C₅₀ aryl)pentarenyl group, a (C₂₂-C₅₀ aryl)indenyl group, a (C₂₁-C₅₀aryl)naphthyl group, a (C₁₉-C₅₀aryl)biphenylenyl group, a (C₇-C₅₀aryl)anthryl group, a (C₂₁-C₅₀aryl)azulenyl group, a (C₁₉-C₅₀aryl)heptalenyl group, a (C₁₉-C₅₀ aryl)acenaphthylenyl group, a (C₁₈-C₅₀ aryl)phenarenyl group, a (C₁₈-C₅₀ aryl)fluolenyl group), a (C₁₆-C₅₀ aryl)methylanthryl group, a (C₁₇-C₅₀ aryl)phenanthrenyl group, a (C₁₇-C₅₀ aryl)pyrenyl group, a (C₁₃-C₅₀ aryl)crysenyl group, a (C₉-C₅₀ aryl)pycenyl group, a (C₁₃-C₅₀ aryl)perylenyl group, a (C₉-C₅₀ aryl)pentaphenyl group, a (C₉-C₅₀ aryl)pentacenyl group, a (C₇-C₅₀ aryl)tetraphenylenyl group, a (C₅-C₅₀aryl)hexaphenyl group, a (C₅-C₅₀aryl)hexacenyl group, a (C₅-C₅₀ aryl)rubicenyl group, a (C₇-C₅₀ aryl)coronenyl group, a (C₅-C₅₀ aryl)trinaphthylenyl group, a (C₅-C₅₀ aryl)heptaphenyl group, a (C₅-C₅₀ aryl)heptacenyl group, a (C₁₈-C₅₀ aryl)fluorenyl group, a (C₅-C₅₀ aryl)pyranthrenyl group, a (C₅-C₅₀ aryl)ovarenyl group and derivatives thereof.

The term “derivatives” used in the present application refers to a group in which at least one hydrogen atom of the groups described above is substituted with the substituents described above. Of those groups described above, Ar₁ may be a 7,12-diphenylbenzo[k]fluoranthenyl group, a (pentaphenyl)phenyl group, a 9,9-di(1-biphenyl)fluorenyl group, a 7,8,9,10-tetraphenylfluoranthenyl group, a 9-(4′-biphenyl-4-yl)-10-phenylanthryl group, a 5,6,11,12-tetraphenylnaphthacenyl group, a 9,10-di(2-naphthyl)anthracenyl group, a 4-(1-biphenyl)-1,1-binaphthyl group, a 6-(terphenyl)chrysenyl group, a 4-sexyphenyl group, a ter(9,9-di(C₆-C₅₀ aryl)fluorenyl group, a 2,7-bis(biphenylene)-9,9-di(C₆-C₅₀ aryl)fluorenyl group, a 12-(biphenyl-2-yl)benzo[j]fluoranthenyl group.

More specifically, R₁ and R₂ may be each independently selected from the group consisting of a hydrogen atom, a C₁-C₅₀ alkyl group, a C₁-C₅₀ alkoxy group, a phenyl group, a tolyl group, a biphenyl group, a pentarenyl group, an indenyl group, a naphthyl group, a biphenylenyl group, an anthryl group, an azulenyl group, a heptalenyl group, an acenaphthylenyl group, a phenarenyl group, a fluorenyl group, a methylanthryl group, a phenanthrenyl group, a triphenylenyl group, a pyrenyl group, a chrysenyl group, an ethyl-crysenyl group, a pycenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovarenyl group, a carbazolyl group, a thiophenyl group, an indolyl group, a purinyl group, a benzimidazolyl group, a quinolinyl group, a benzothiophenyl group, a parathiazinyl group, a pyrolyl group, a pyrazolyl group, an imidazolyl group, an imidazolinyl group, a oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxyranyl group, a pyrrolidinyl group, a pyrazolidinyl group, an imidazolidinyl group, a piperidinyl group, a piperazinyl group, a morpolinyl group, a di(C₆-C₅₀ aryl)amino group, a di(C₆-C₅₀ aryl)aminophenyl group, a tri(C₆-C₅₀ aryl)silyl group and derivatives thereof.

Of those groups described above, R₁ and R₂ may be each independently a methyl group, a methoxy group, a phenyl group, a tolyl group, a naphthyl group, an anthracenyl group, a pyrenyl group, a phenanthrenyl group, a fluorenyl group, an imidazolynyl group, an indolyl group, a quinolinyl group, a diphenylamino group, a 2,3-di-p-tolylaminophenyl group, a naphthylphenylamino group, a dinaphthylamino group and a triphenylsilyl group.

The organic light emitting compound can be advantageously represented by one of Formulae 4 through 19 below, but is not limited thereto:

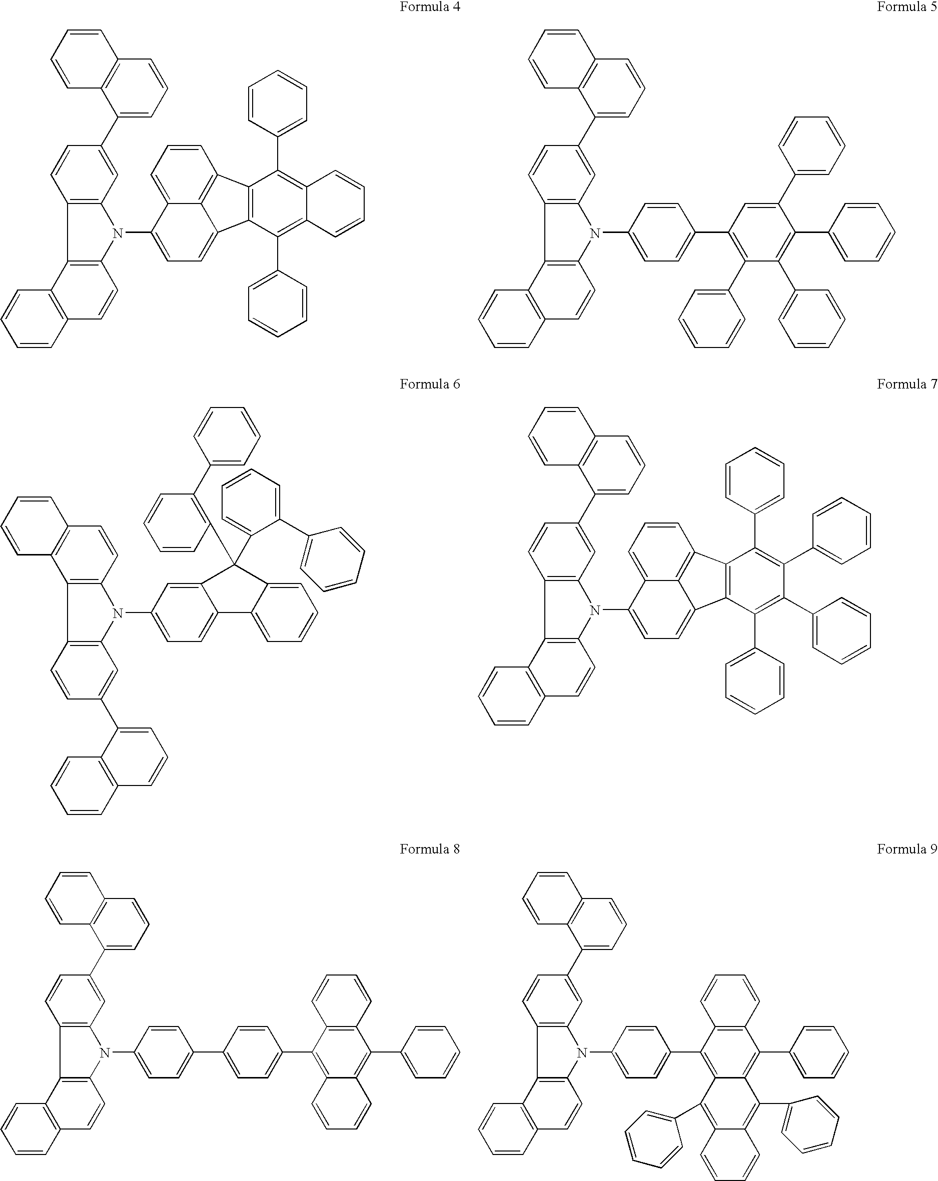

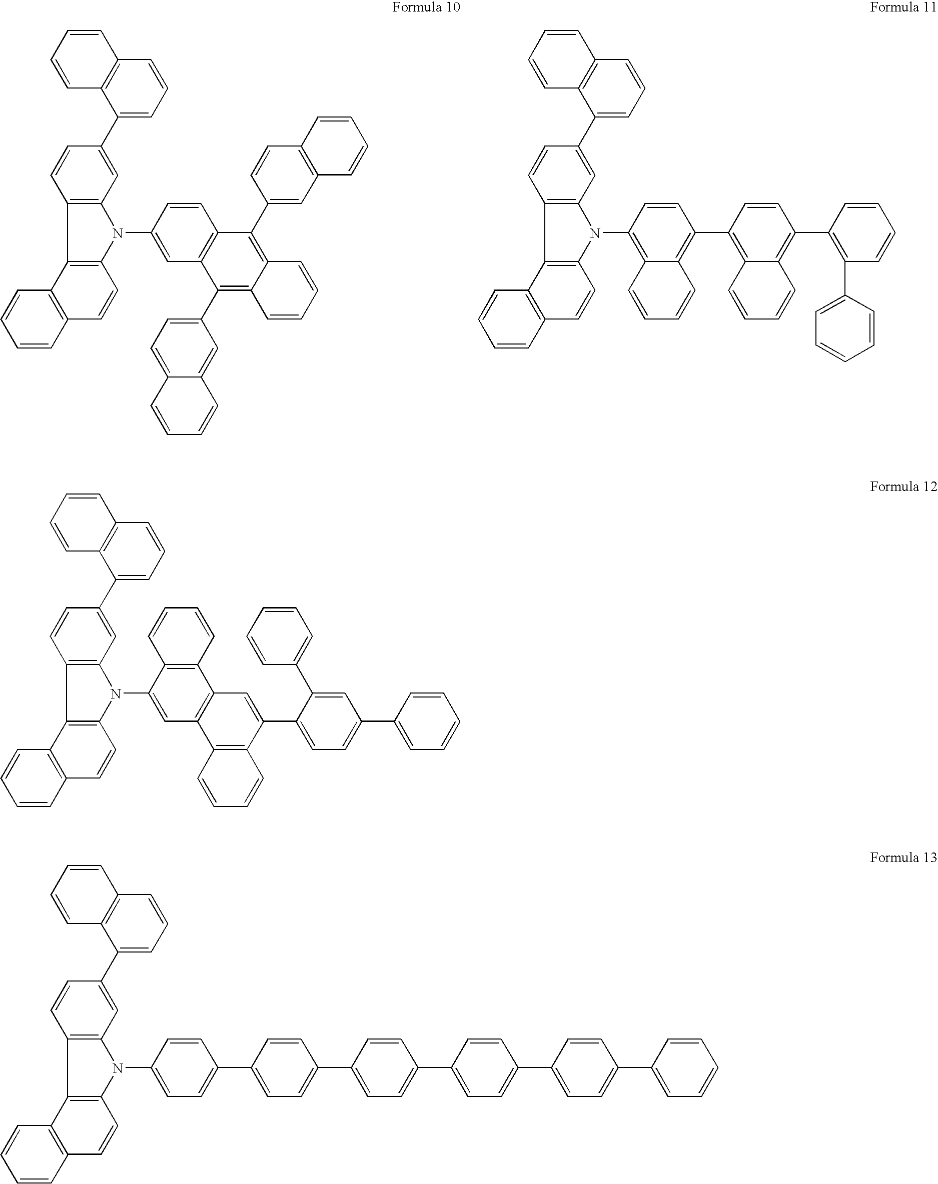

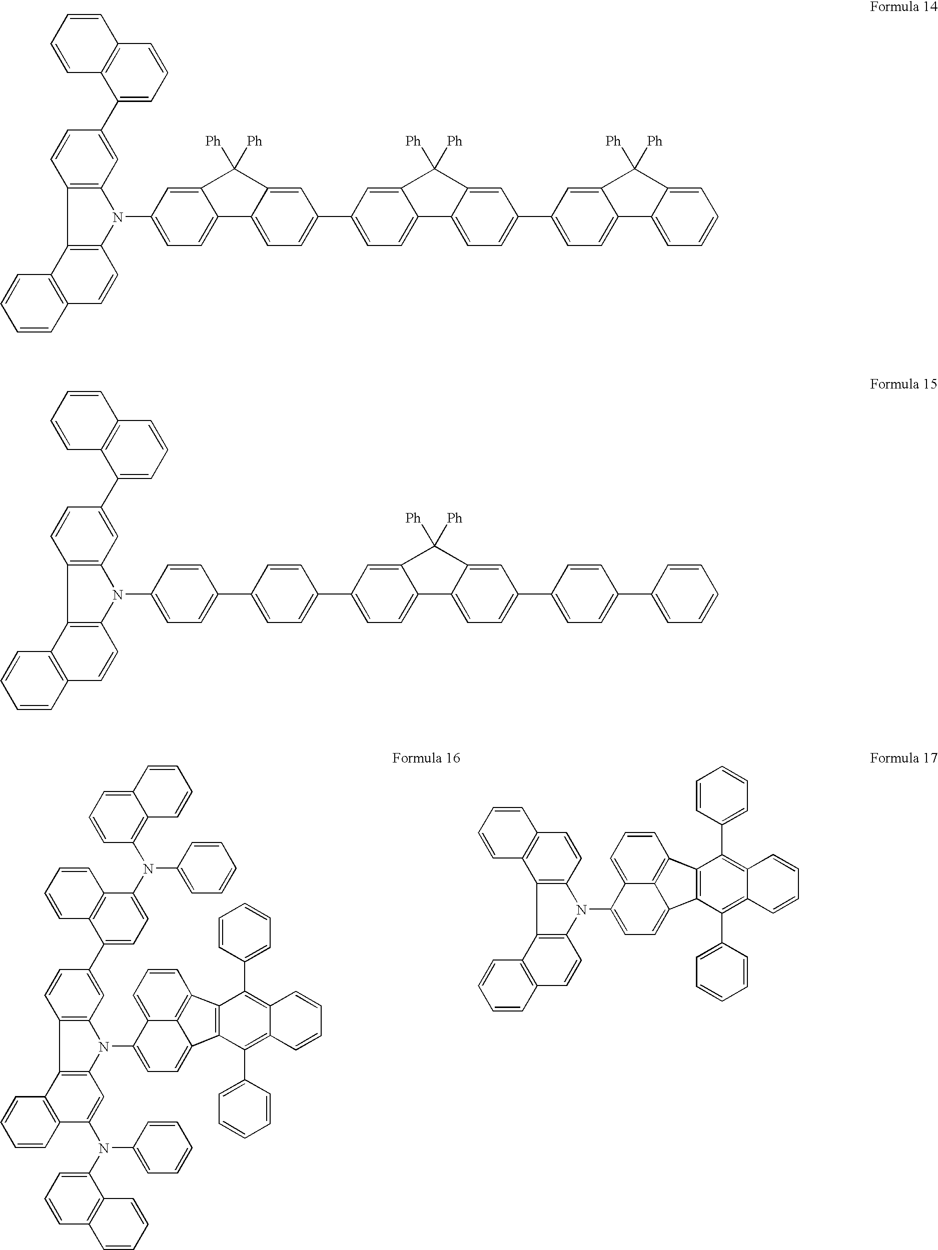

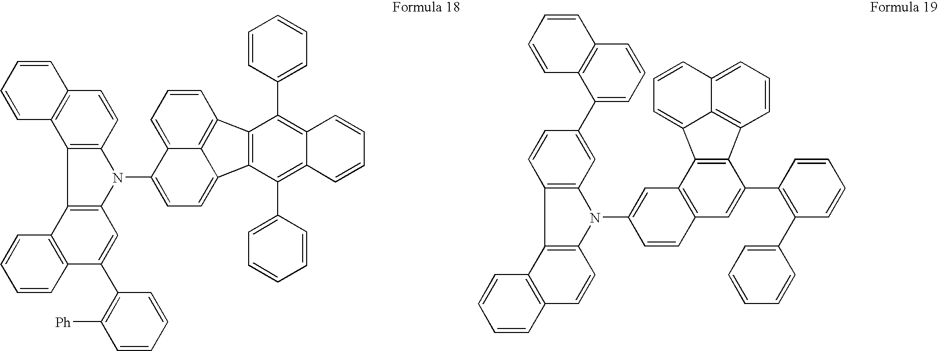

The organic light emitting compound represented by any one of Formulae 4 through 19 can be synthesized using conventional synthesis methods which will be described in more detail below with reference to reaction schemes of Synthesis Examples.

Thermal stability of the organic light emitting compounds represented by Formulae 1 through 19 can be determined by measuring the glass transition temperature (Tg) and the melting point (Tm) of the compounds through thermal analysis using thermo gravimetric analysis (TGA) and differential scanning calorimetry (DSC). For example, degradation temperature and Tg of the compound represented by Formula 4 are 470° C. and 215° C., respectively. From the results, it can be seen that an organic light emitting compound having high thermal stability can be obtained using any of the organic light emitting compounds represented by Formulae 1 through 19. In addition, emitting ability of each of the organic light emitting compounds represented by Formulae 1 through 19 can be evaluated by measuring PL spectra of the compounds. For example, the compound represented by Formula 4 has a maximum wavelength of 445 nm in solution, and the Commission Internationale de L'Eclairage (CIE) coordinate thereof is (0.15, 0.10). From the results, it can be seen that the organic light emitting compounds represented by Formulae 1 through 19 are materials that emit blue-light, and have high color purity.

According to an embodiment, there is provided an OLED comprising: a first electrode; a second electrode; and an organic layer interposed between the first electrode and the second electrode, wherein the organic layer comprises at least one of the organic light emitting compounds represented by Formulae 1 through 3.

The organic light emitting compounds represented by Formulae 1 through 3 are suitable for an organic layer of the OLED, specifically, an emission layer, a hole injection layer or a hole transport layer.

Unlike a conventional OLED which comprises an organic layer having low stability, the OLED described herein can have a low turn-on voltage, a high color purity, and the like, by comprising an organic light emitting compound that has good solubility and high thermal stability, and can form a stable organic layer, when manufactured using the solution coating method.

The OLED can have various structures, and can further comprise at least one layer selected from the group consisting of a hole injection layer, a hole transport layer, a hole blocking layer, an electron blocking layer, an electron transport layer and an electron injection layer, between the first electrode and the second electrode.

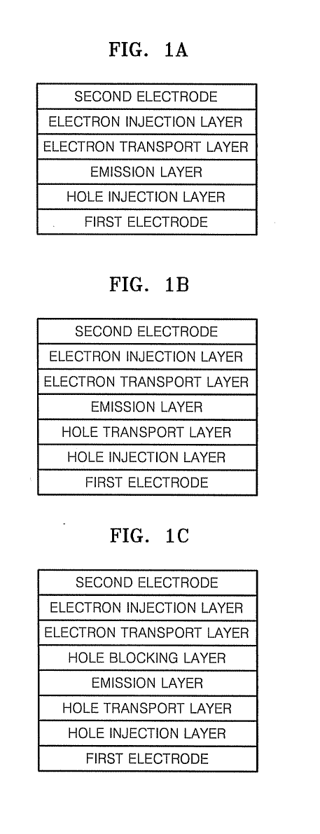

More specifically, FIGS. 1A through 1C are schematic cross-sectional views illustrating exemplary structures of the OLED according to an embodiment. Referring to FIG. 1A, the OLED has a first electrode/hole injection layer/emission layer/electron transport layer/electron injection layer/second electrode structure. Referring to FIG. 1B, the OLED has a first electrode/hole injection layer/hole transport layer/emission layer/electron transport layer/electron injection layer/second electrode structure. Referring to FIG. 1C, the OLED has a first electrode/hole injection layer/hole transport layer/emission layer/hole blocking layer/electron transport layer/electron injection layer/second electrode structure. At least one of the emission layer, the hole injection layer and the hole transport layer can comprise the organic light emitting compound. The emission layer may comprise a red, green, blue or white phosphorescent or fluorescent dopant. The phosphorescent dopant can be an organometallic compound comprising at least one of Ir, Pt, Os, Ti, Zr, Hf, Eu, Tb, and Tm. Further, the organic light emitting compound can be used as a fluorescent dopant on the emission layer.

Hereinafter, a method of manufacturing an OLED according to an embodiment will be described with reference to the OLED illustrated in FIG. 1C.

First, a first electrode is formed by disposing a high work-function material on a substrate using, for example, a deposition of a sputtering technique. The first electrode can be an anode. The substrate, which can be any substrate that is used in conventional OLEDs, may be a glass substrate or a transparent plastic substrate that has excellent mechanical strength, thermal stability, transparency, and surface smoothness, can be easily treated, and is waterproof. The first electrode can be formed of indium tin oxide (ITO), indium zinc oxide (IZO), tin oxide (SnO₂), zinc oxide (ZnO), or the like, or a combination comprising at least one of the foregoing oxides.

Then, a hole injection layer (HIL) can be formed on the first electrode by vacuum deposition, spin coating, casting, Langmuir Blodgett (LB) deposition, or the like, or a combination comprising at least one of the foregoing methods.

When the HIL is formed by vacuum deposition, vacuum deposition conditions may vary according to the compound that is used to form the HIL, and the desired structure and thermal properties of the HIL to be formed. In general, however, the vacuum deposition may be performed at a deposition temperature of about 100° C. to about 500° C., under a pressure of about 10⁻⁸ to about 10⁻³ torr (about 1.33×10⁻⁹ to about 1.33×10⁻⁴ kPa), at a deposition speed of about 0.01 to about 100 Å/sec, and to a layer thickness of about 100 Å to about 10 μm.

When the HIL is formed by spin coating, coating conditions may vary according to the compound that is used to form the HIL, and the desired structure and thermal properties of the HIL to be formed. In general, however, the coating speed may be in the range of about 2,000 to about 5,000 rpm, and the temperature for heat treatment, which is performed to remove the solvent after coating, may be in the range of about 80 to about 200° C.

The material used to form the HIL can be formed of the organic light emitting compound represented by Formula 1. For example, the material may be a phthalocyanine compound, such as copper phthalocyanine disclosed in U.S. Pat. No. 4,356,429; a star-burst type amine derivative, such as TCTA, m-MTDATA, or m-MTDAPB, disclosed in Advanced Material, 6, p. 677 (1994); soluble and conductive polymer such as polyaniline/Dodecylbenzenesulfonic acid (PANI/DBSA); poly(3,4-ethylenedioxythiophene)/poly(4-styrenesulfonate) (PEDOT/PSS): polyaniline/camphor sulfonic acid (Pani/CSA); (polyaniline)/poly(4-styrenesulfonate) (PANI/PSS); or the like.

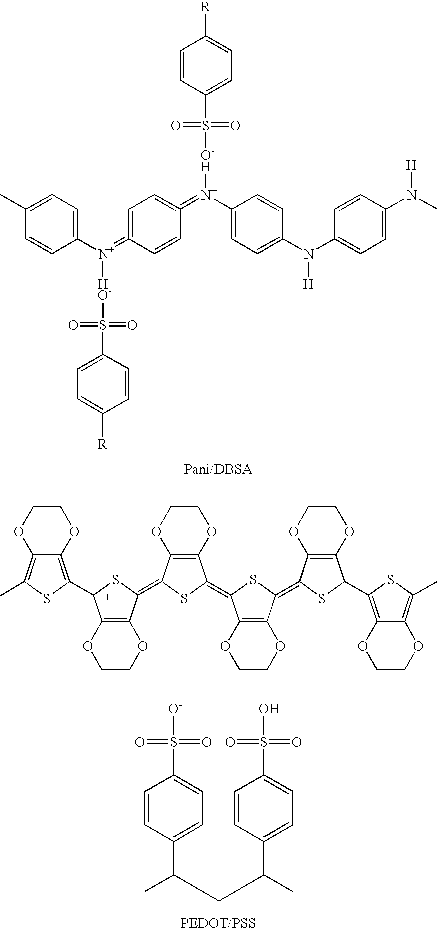

The thickness of the HIL may be an amount of about 100 to about 10,000 Å, and preferably, an amount of about 100 to about 1,000 Å. When the thickness of the HIL is less than about 100 Å, the hole injecting ability of the HIL may be reduced. On the other hand, when the thickness of the HIL is greater than about 10,000 Å, the turn-on voltage of the OLED can be increased.

Then, a hole transport layer (HTL) can be formed on the HIL using vacuum deposition, spin coating, casting, LB, or the like, or a combination comprising at least one of the foregoing methods. When the HTL is formed by vacuum deposition or spin coating, the deposition and coating conditions are similar to those for the formation of the HIL, although the deposition and coating conditions may vary according to the material that is used to form the HTL.

The material used to form the HTL can comprise the organic light emitting compound represented by Formula 1 described above. In addition, for example, the HTL can be formed of a carbazole derivative, such as N-phenylcarbazole, polyvinylcarbazole; a typical amine derivative having an aromatic condensation ring such as N,N′-bis(3-methylphenyl)-N,N′-diphenyl-[1,1-biphenyl]-4,4′-diamine(TPD), N,N′-di(naphthalene-1-yl)-N,N′-diphenyl benzydine (α-PD); or the like. The thickness of the HTL may be in the range of about 50 to about 1,000 Å, and preferably, about 100 to about 600 Å. When the thickness of the HTL is less than about 50 Å, the hole transporting ability of the HTL may be reduced. On the other hand, when the thickness of the HTL is greater than about 1,000 Å, the turn-on voltage of the OLED may increase.

Then, an emission layer (EML) can be formed on the HTL by vacuum deposition, spin coating, casting, LB deposition, or the like. When the EML is formed by vacuum deposition or spin coating, the deposition and coating conditions are similar to those for the formation of the HIL, although the deposition and coating conditions may vary according to the material that is used to form the EML.

The emission layer can comprise the organic light emitting compound represented by Formula 1 described above. The emission layer can be formed using a known host material or a known dopant material in addition to the compound of Formula 1. The organic light emitting compound of Formula 1 can be used alone. The host material may be, for example, tris-(8-hydroxyquinoline) aluminum (Alq₃), 4,4′-N,N′-dicarbazole-biphenyl (CBP), poly(n-vinylcarbazole) (PVK), or the like.

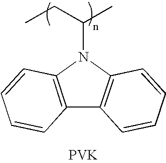

As for the dopant material, examples of a fluorescent dopant include, but are not limited to, IDE102 and IDE105 obtained from Idemitsu Co., C545T obtained from Hiyasibara Co., and the like, and examples of a phosphorescent dopant include ared phosphorescent dopant such as platinum (II) octaethyl porphine (PtOEP), or RD 61, obtained from UDC Co., a green phosphorescent dopant such as Ir(PPy)₃ (PPy=2-phenylpyridine), a blue phosphorescent dopant such as bis[2-(4,6-difluorophenyl)pyridinato-N,C2′] iridium picolinate (F2Irpic) obtained from UDC Co., and the like.

The concentration of the dopant is not limited, but is advantageously in the amount of about 0.01 to about 15 parts by weight based on 100 parts by weight of a host.

The thickness of the EML may be in the range of about 100 to about 1,000 Å, and preferably, in the range of about 200 to about 600 Å. When the thickness of the EML is less than about 100 Å, the emissive ability of the EML may be reduced. On the other hand, when the thickness of the EML is greater than about 1,000 Å, the turn-on voltage of the OLED may increase. When the EML comprises a phosphorous dopant, a hole blocking layer (HBL) can be formed on the EML by vacuum deposition, spin coating, casting, LB deposition, or the like, in order to prevent triplet excitons or holes from migrating into an electron transport layer (ETL). When the HBL is formed by vacuum deposition or spin coating, the deposition and coating conditions are similar to those for the formation of the HIL, although deposition and coating conditions may vary according to the material that is used to form the HBL. Examples of the material used to form the HBL include oxadiazole derivatives, triazole derivatives, phenanthroline derivatives such as 2,9-dimethyl-4,7-diphenyl-1,10-phenanthroline (BCP), and the like.

The thickness of the HBL may be in the range of about 50 to about 1,000 Å, and preferably, in the range of about 100 to about 300 Å. When the thickness of the HBL is less than about 50 Å, the hole blocking ability of the HBL may be reduced. On the other hand, when the thickness of the HBL is greater than about 1,000 Å, the turn-on voltage of the OLED may increase.

Then, an ETL is formed by vacuum deposition, spin coating, casting, or the like. When the ETL is formed by vacuum deposition or spin coating, the deposition and coating conditions are, in general, similar to those for the formation of the HIL, although the deposition and coating conditions may vary according to the material that is used to form the ETL. The ETL transports electrons injected from the cathode, and the ETL may be formed of a quinoline derivative such as Alq₃, 2-(biphenyl-4-yl)-5-(4-tert-butylphenyl)-1,3,4-oxadiazole, 3-(biphenyl-4-yl)-4-phenyl-5-(4-tert-butylphenyl)-1,2,4-triazole (TAZ), Balq or the like.

The thickness of the ETL may be in the range of about 100 to about 1,000 Å, and preferably, about 200 to about 500 Å. When the thickness of the ETL is less than about 100 Å, the electron transporting ability of the ETL may be reduced. On the other hand, when the thickness of the ETL is greater than about 1,000 Å, the turn-on voltage of the OLED may increase.

In addition, an electron injection layer (EIL) that makes it easy for electrons to be injected from a cathode may be formed on the ETL. There are no limits as to the material used to form the EIL.

The EIL may be formed of lithium fluoride (LiF), sodium chloride (NaCl), cesium fluoride (CsF), lithium oxide (Li₂O), barium oxide (BaO), or the like or a combination comprising at least one of the foregoing. Conditions for the deposition of the EIL are, in general, similar to conditions for the formation of the HIL, although they may vary according to the material that is used to form the EIL.

The thickness of the EIL may be in the range of about 1 to about 100 Å, and preferably, about 5 to about 50 Å. When the thickness of the EIL is less than about 1 Å, the electron injecting ability of the EIL may be reduced. On the other hand, when the thickness of the EIL is greater than about 100 Å, the turn-on voltage of the OLED may increase. Finally, a second electrode can be formed on the EIL by vacuum deposition, sputtering, or the like. The second electrode can be used as a cathode. The second electrode may be formed of a low work-function metal, an alloy, an electrically conductive compound, or a combination thereof. In particular, the second electrode may be formed of lithium (Li), magnesium (Mg), aluminum (Al), aluminum-lithium (Al—Li), calcium (Ca), magnesium-indium (Mg—In), magnesium-silver (Mg—Ag), or the like. Alternatively, a transparent cathode formed of ITO or IZO can be used to produce a front surface LED.

A method of manufacturing the OLED according to an embodiment comprises: forming a first electrode; forming an organic thin film comprising the organic light emitting compound represented by any one of Formulae 1 through 19 on the first electrode; and forming a second electrode on the organic thin film. A step of sintering the organic thin film can be performed before forming the second electrode. The organic thin film may be formed using a wet spinning method including deposition, spin coating, inkjet printing and spray printing or a heat transfer method.

Hereinafter, Synthesis Examples and Examples of organic light emitting compounds according to embodiments of the present invention will be described in detail. However, the Synthesis Examples and Examples are provided to facilitate the understanding of the present invention only, and are not intended to limit the scope of the present invention.

### EXAMPLES

### Synthesis Example 1

Compound 4 represented by Formula 4 was synthesized through Reaction Schemes 1, 2 and 3:

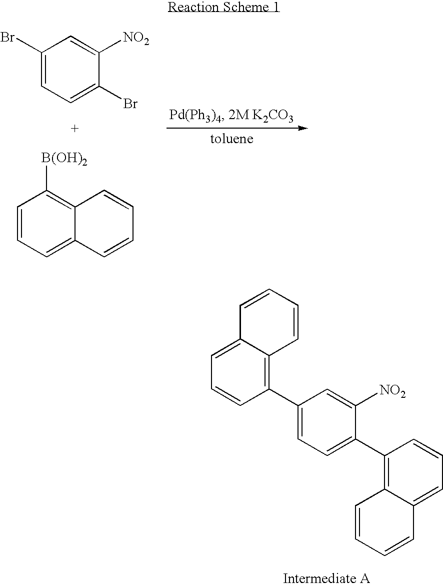

### Synthesis of Intermediate A

8.4 g of 2,5-dibromonitrobenzene (30 mmol), 10.8 g of 1-naphthaleneboronic acid (62.6 mmol), 520 mg of tetrakis triphenylphosphine palladium (Pd(PPh₃)₄) (0.45 mmol) and 63 ml of a 2M aqueous potassium carbonate solution (126 mmol) were dissolved in 100 ml of toluene, respectively, and then the mixtures were added to a 500 ml round bottom flask. Then, the mixture was refluxed for 24 hours. After the reaction was terminated, the solvent was removed by evaporation. Then, the residue washed with 500 ml of ethylacetate and 500 ml of water. Thereafter, the organic layer was collected and was dried with anhydrous magnesium sulfate. Subsequently, the dried organic layer was purified with silica chromatography to obtain 9.5 g of a compound represented by Intermediate A (yield 84%).

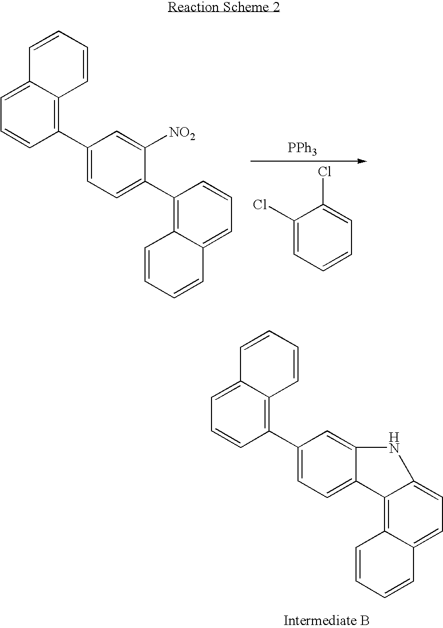

### Synthesis of Intermediate B

8.0 g of Intermediate A (21.3 mmol) and 14 g of triphenylphosphine (PPh₃) (53.3 mmol) were dissolved in 42 ml of 1,2-dichlorobenzene, and the mixture was added to a 500 ml round bottom flask and then refluxed for 24 hours. After the reaction was terminated, the reactant was purified with silica chromatography to obtain 4.1 g of a compound represented by Intermediate B (yield 56%).

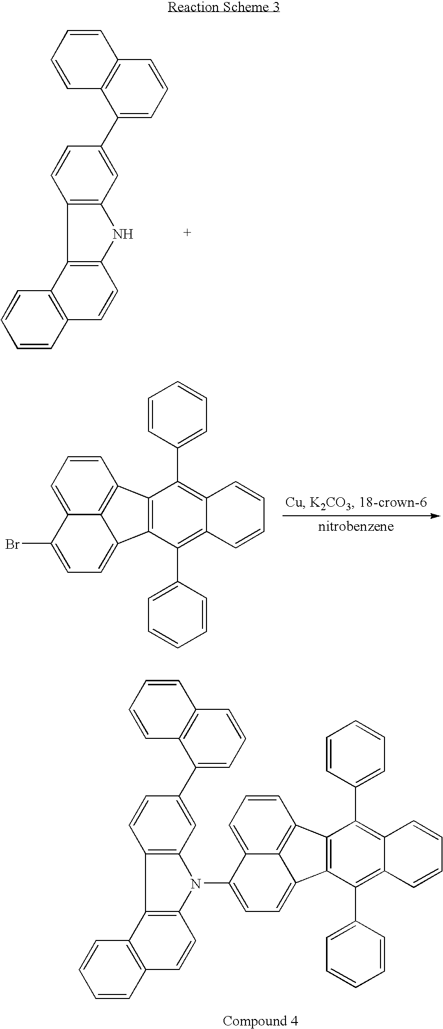

### Synthesis of Compound 4

546 mg of Intermediate B (1.6 mmol), 202 mg of copper (3.2 mmol), 879 mg of potassium carbonate (6.4 mmol), 126 mg of 18-crown-6 (0.48 mmol), 1.0 g of 4-bromo-(7,12-diphenyl)benzo[k]fluoranthene (2.1 mmol) were dissolved in 5 ml of nitrobenzene, and the mixture was added to a 500 ml round bottom flask and then refluxed for 24 hours. After the reaction was terminated, a solvent was removed by evaporation. Then, the residue washed with 50 ml of ethylacetate and 50 ml of water. Thereafter, an organic layer was collected and was dried with anhydrous magnesium sulfate. Subsequently, the dried organic layer was purified with silica chromatography to obtain 400 mg of a compound represented by Compound 4 (yield 34%).

¹H-NMR (CDCl₃, 300 MHz, ppm): 8.9-6.5 (m, 35H).

### Synthesis Example 2

Compound 8 represented by Formula 8 was synthesized through Reaction Scheme 4:

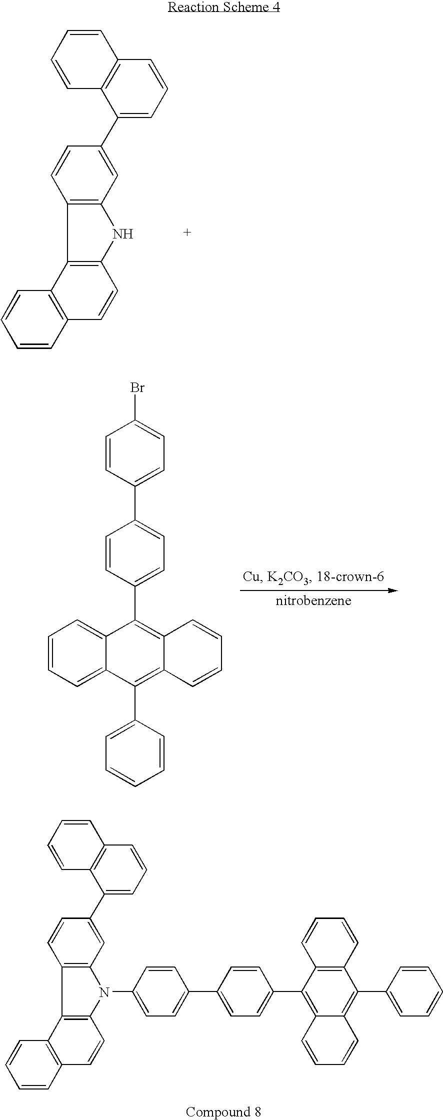

### Synthesis of Compound 8

1.1 g of Intermediate B (3.2 mmol), 405 mg of copper (6.4 mmol), 1.8 g of potassium carbonate (12.8 mmol), 250 mg of 18-crown-6 (1 mmol), 970 mg of 9-(4-bromobiphenyl-4-yl)-10-phenylanthracene (2.2 mmol) were dissolved in 10 ml of nitrobenzene, and the mixture was added to a 500 ml round bottom flask and then refluxed for 24 hours. After the reaction was terminated, the solvent was removed by evaporation. Then, the residue washed with 100 ml of ethylacetate and 100 ml of water. Thereafter, the organic layer was collected and was dried with anhydrous magnesium sulfate. Subsequently, the dried organic layer was purified with silica chromatography to obtain 674 mg of a compound represented by Compound 8 (yield 41%).

¹H-NMR (CDCl₃, 300 MHz, ppm): 8.9-7.3 (m, 37H).

### Synthesis Example 3

Compound 17 represented by Formula 17 was synthesized through Reaction Scheme 5 below:

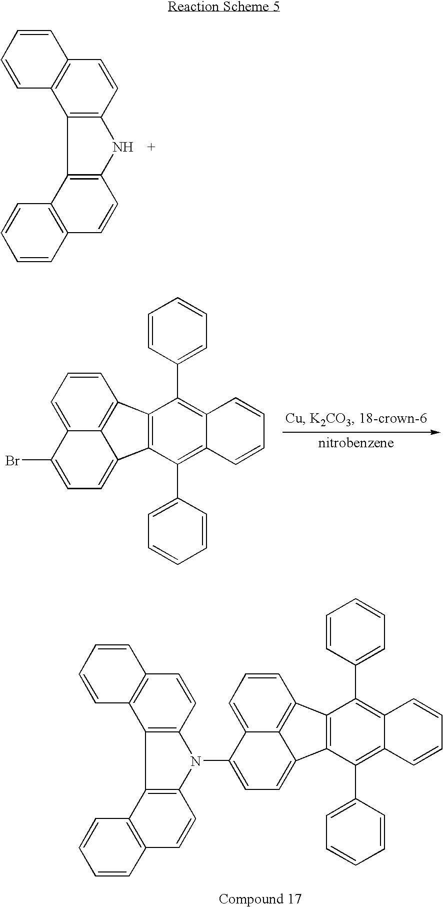

### Synthesis of Compound 17

427 mg of 7H-dibenzo[c,g]carbazole (1.6 mmol), 202 mg of copper (3.2 mmol), 879 mg of potassium carbonate (6.4 mmol), 126 mg of 18-crown-6 (0.48 mmol), 1.0 g of 4-bromo-(7,12-diphenyl)benzo[k] fluoranthene (2.1 mmol) were dissolved in 5 ml of nitrobenzene, and the mixture was added to a 500 ml round bottom flask and then refluxed for 24 hours. After the reaction was terminated, the solvent was removed by evaporation. Then, the residue washed with 50 ml of ethylacetate and 50 ml of water. Thereafter, the organic layer was collected and was dried with anhydrous magnesium sulfate. Subsequently, the dried organic layer was purified with silica chromatography to obtain 268 mg of a compound represented by Compound 17 (yield 25%).

¹H-NMR (CDCl₃, 300 MHz, ppm): 8.9-6.5 (m, 31H).

### Evaluation Example 1

### Evaluation of Emitting Ability of Compound (Solution State)

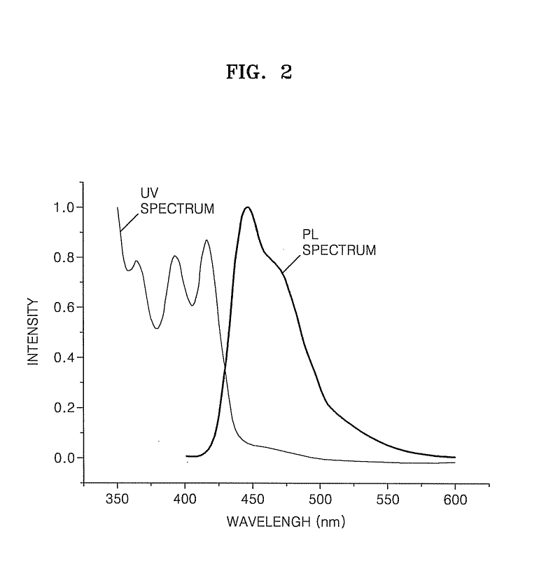

Emitting ability of each compound was evaluated by measuring their PL spectra. First, Compound 4 was diluted to a concentration of 10 mM in toluene. Then, the PL spectrum was measured using an ISC PC1 spectrofluorometer in which a Xenon lamp was installed. These processes were repeated with respect to Compounds 8 and 17. The results are shown in Table 1 below. The experimental result of Compound 4 is shown in FIG. 2.

TABLE 1

| Compound No. | PL wavelength (nm) |
|---|---|
| 4 | 445 |
| 8 | 445 |
| 17 | 460 |

From the results, it can be seen that organic light emitting compounds according to embodiments of the present invention have emitting abilities suitable for an organic light emitting device.

### Example 1

Using Compound 4 as a dopant of an emission layer, an organic light emitting device having the following structure was manufactured: ITO/α-NPD (500 Å)/Compound 4+ADN(500 Å)/Alq₃(200 Å)/LiF(10 Å)/Al(2000 Å). As an anode, an ITO glass substrate having an electrical resistance of 15 ohm per square centimeter (Ω/cm²) at a thickness of 1000 Å was cut to a size of 50 mm×50 mm×0.7 mm, microwave washed with isopropyl alcohol and pure water for 15 minutes each, respectively, and then washed with UV ozone for 30 minutes. α-NPD was vacuum deposited on the substrate to form a HIL with a thickness of 500 Å. Compound 4 and 9,10-di(naphthalene-2-yl)anthracene (ADN) (2 volume parts of Compound 4 based on 100 volume parts of ADN 100) were vacuum deposited on the HIL to form an EML with a thickness of 500 Å. Then, Alq₃ was vacuum deposited on the EML to form an ETL with a thickness of 200 Å. 10 Å of LiF and 2000 Å of Al were sequentially vacuum deposited on the ETL to form an EIL and a cathode, respectively. Accordingly, an OLED illustrated in FIG. 1A was manufactured. The device is referred to as sample 1.

### Example 2

An OLED having the structure ITO/α-PD(500 Å)/Compound 8+ADN(500 Å)/Alq₃(200 Å)/LiF(10 Å)/Al(2000 Å) was manufactured in the same manner as in Example 1, except that Compound 8 was used as the dopant instead of Compound 4. The device is referred to as sample 2.

### Example 3

An OLED having the structure ITO/α-NPD(500 Å)/Compound 17+ADN(500 Å)/Alq₃(200 Å)/LiF(10 Å)/Al(2000 Å) was manufactured in the same manner as in Example 1, except that Compound 17 was used as the dopant instead of Compound 4. The device is referred to as sample 3.

### Comparative Example 1

An OLED having the structure of ITO/α-PD (500 Å)/Compound 21+ADN(500 Å)/Alq₃(200 Å)/LiF(10 Å)/Al(2000 Å) was manufactured in the same manner as in Example 1, except that Compound 21 was used as the dopant instead of Compound 4. The device is referred to as sample 4. Compound 21 is represented by the Formula 21 below.

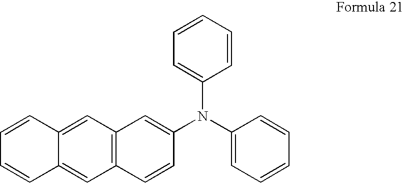

### Evaluation Example 4

### Evaluation of Properties of Samples 1, 2, 3 and 4

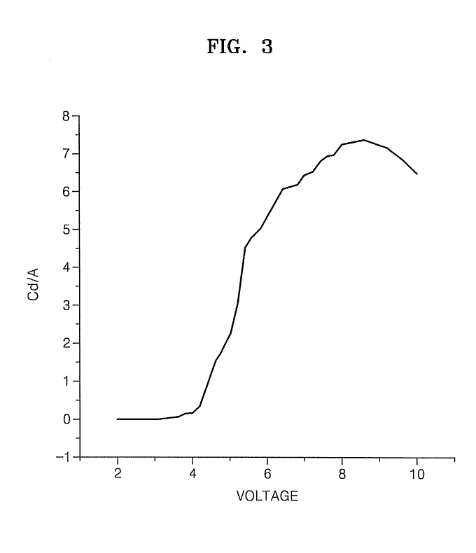

Turn-on voltage, efficiency and luminance of Samples 1, 2 and 3 and comparative Sample 4 were measured using a PR650 (Spectroscan) Source Measurement Unit, respectively. The results are shown in Table 2 below. The electrical efficiency curve of sample 1 is shown in FIG. 3.

TABLE 2

| Sample | Turn-on | Efficiency | Luminance |
|---|---|---|---|
| No. | Voltage (V) | (cd/A) | (cd/m²) |
| 1 | 3.2 | 7.4 | 8159 |
| 2 | 3.2 | 5.5 | 7221 |
| 3 | 3.2 | 7.2 | 7821 |
| 4 | 3.4 | 3.1 | 4500 |

From the results shown in Table 2, it can be seen that Samples 1 through 3 have good electrical properties.

The organic light emitting compounds represented by Formulae 1 through 3 have good solubility, good emitting ability and high thermal stability. Therefore, when the organic light emitting compounds represented by Formulae 1 through 19 are used, an OLED having low turn-on voltage, high efficiency and high luminance can be manufactured.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An organic light emitting compound represented by Formula 1: 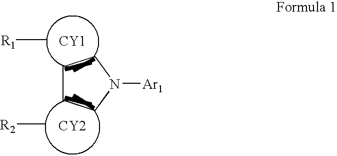
wherein CY1 and CY2 are each independently a benzene ring or a naphthalene ring; Ar₁ is a substituted or unsubstituted C₃₁-C₁₀₀ aryl group; R₁ and R₂ are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group; with the proviso that CY1 and CY2 are not both benzene rings.

2. The organic light emitting compound of claim 1, wherein the compound is represented by Formula 2: 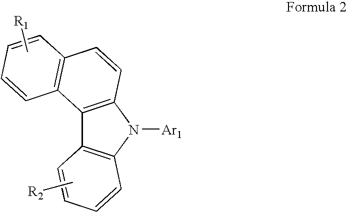
wherein Ar₁ is a substituted or unsubstituted C₃₁-C₁₀₀aryl group; and R₁ and R₂ are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group.

3. The organic light emitting compound of claim 1, wherein the compound is represented by Formula 3: 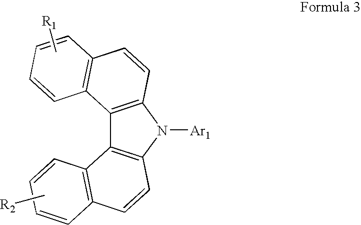
wherein Ar₁ is a substituted or unsubstituted C₃₁-C₁₀₀aryl group; and R₁ and R₂ are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group.

4. The organic light emitting compound of claim 1, wherein substituents of the alkyl group, the alkoxy group, the aryl group, the heteroaryl group, the cycloalkyl group and the heterocycloalkyl group are selected from the group consisting of —F; —Cl; —Br; —CN; —NO₂; —OH; an unsubstituted C₁-C₅₀ alkyl group or a C₁-C₅₀ alkyl group substituted with —F, —Cl, —Br, —CN, —NO₂ or —OH; an unsubstituted C₁-C₅₀ alkoxy group or a C₁-C₅₀ alkoxy group substituted with —F, —Cl, —Br, —CN, —NO₂ or —OH; an unsubstituted C₆-C₅₀ aryl group or a C₆-C₅₀ aryl group substituted with a C₁-C₅₀ alkyl group, a C₁-C₅₀ alkoxy group, —F, —Cl, —Br, —CN, —NO₂ or —OH; an unsubstituted C₂-C₅₀ heteroaryl group or a C₂-C₅₀ heteroaryl group substituted with a C₁-C₅₀ alkyl group, a C₁-C₅₀ alkoxy group, —F, —Cl, —Br, —CN, —NO₂ or —OH; an unsubstituted C₅-C₅₀ cycloalkyl group or a C₅-C₅₀ cycloalkyl group substituted with a C₁-C₅₀ alkyl group, a C₁-C₅₀ alkoxy group, —F, —Cl, —Br, —CN, —NO₂ or —OH; an unsubstituted C₅-C₅₀ heterocycloalkyl group or a C₅-C₅₀ heterocycloalkyl group substituted with a C₁-C₂₀ alkyl group, a C₁-C₂₀ alkoxy group, —F, —Cl, —Br, —CN, —NO₂ or —OH; and —N(Z₉)(Z₁₀) where Z₉ and Z₁₀ may be each independently a hydrogen atom, a C₁-C₅₀ alkyl group, or a C₆-C₅₀ aryl group substituted with a C₁-C₅₀ alkyl group.

5. The organic light emitting compound of claim 1, wherein Ar₁ is selected from the group consisting of a di(C₆-C₅₀aryl)benzo[k]fluoranthenyl group, a (C₁₁-C₅₀ aryl)benzo[k]fluoranthenyl group, a (pentaC₆-C₅₀ aryl)phenyl group, a (C₁₃-C₅₀ aryl)terphenyl group, a 9,9-di(C₁₂-C₅₀ aryl)fluorenyl group, a (C₁₅-C₅₀ aryl)fluoranthenyl group, a 9-(C₆-C₅₀aryl)-10-(C₁₁-C₅₀aryl)anthryl group, a (C₁₃-C₅₀ aryl)naphthacenyl group, a (C₁₇-C₅₀ aryl)anthryl group, a ((C₁₁-C₅₀ aryl)naphthyl)naphthyl group, a (C₆-C₅₀ aryl)sexyphenyl group, a ter(di(C₆-C₅₀ aryl)fluorenyl group, a bis(biphenylene)-9,9-di(C₆-C₅₀aryl)fluorenyl group, a (C₁₁-C₅₀ aryl)benzo[j]fluoranthenyl group, a (C₂₃-C₅₀aryl)pentarenyl group, a (C₂₂-C₅₀aryl)indenyl group, a (C₂₁-C₅₀aryl)naphthyl group, a (C₁₉-C₅₀aryl)biphenylenyl group, a (C₇-C₅₀aryl)anthryl group, a (C₂₁-C₅₀aryl)azulenyl group, a (C₁₉-C₅₀aryl) heptalenyl group, a (C₁₉-C₅₀ aryl)acenaphthylenyl group, a (C₁₈-C₅₀ aryl)phenarenyl group, a (C₁₈-C₅₀ aryl)fluorenyl group), a (C₁₆-C₅₀ aryl)methylanthryl group, a (C₁₇-C₅₀ aryl)phenanthrenyl group, a (C₁₇-C₅₀ aryl)pyrenyl group, a (C₁₃-C₅₀ aryl)chrysenyl group, a (C₉-C₅₀ aryl)pycenyl group, a (C₁₃-C₅₀ aryl)perylenyl group, a (C₉-C₅₀ aryl)pentaphenyl group, a (C₉-C₅₀ aryl)pentacenyl group, a (C₇-C₅₀ aryl)tetraphenylenyl group, a (C₅-C₅₀aryl)hexaphenyl group, a (C₅-C₅₀aryl)hexacenyl group, a (C₅-C₅₀ aryl)rubicenyl group, a (C₇-C₅₀ aryl)coronenyl group, a (C₅-C₅₀ aryl)trinaphthylenyl group, a (C₅-C₅₀ aryl)heptaphenyl group, a (C₅-C₅₀ aryl)heptacenyl group, a (C₁₈-C₅₀ aryl)fluorenyl group, a (C₅-C₅₀ aryl)pyranthrenyl group, a (C₅-C₅₀ aryl)ovarenyl group and derivatives thereof.

6. The organic light emitting compound of claim 1, wherein Ar₁ is a 7,12-diphenylbenzo[k]fluoranthenyl group, a (pentaphenyl)phenyl group, a 9,9-di(1-biphenyl)fluorenyl group, a 7,8,9,10-tetraphenylfluoranthenyl group, a 9-(4′-biphenyl-4-yl)-10-phenylanthryl group, a 5,6,11,12-tetraphenylnaphthacenyl group, a 9,10-di(2-naphthyl)anthracenyl group, a 4-(1-biphenyl)-1,1-binaphthyl group, a 6-(terphenyl)chrysenyl group, a 4-sexyphenyl group, a ter(9,9-di(C₆-C₅₀aryl)fluorenyl group, a 2,7-bis(biphenylene)-9,9-di(C₆-C₅₀aryl)fluorenyl group, or a 12-(biphenyl-2-yl)benzo[j]fluoranthenyl group and derivatives thereof.

7. The organic light emitting compound of claim 1, wherein R₁ and R₂ are each independently selected from the group consisting of a hydrogen atom, a C₁-C₅₀ alkyl group, a C₁-C₅₀ alkoxy group, a phenyl group, a tolyl group, a biphenyl group, a pentarenyl group, an indenyl group, a naphthyl group, a biphenylenyl group, an anthryl group, an azulenyl group, a heptalenyl group, an acenaphthylenyl group, a phenarenyl group, a fluorenyl group, a methylanthryl group, a phenanthrenyl group, a triphenylenyl group, a pyrenyl group, a chrysenyl group, an ethyl-chrysenyl group, a pycenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovarenyl group, a carbazolyl group, a thiophenyl group, an indolyl group, a purinyl group, a benzimidazolyl group, a quinolinyl group, a benzothiophenyl group, a parathiazinyl group, a pyrolyl group, a pyrazolyl group, an imidazolyl group, an imidazolynyl group, a oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxyranyl group, a pyrrolidinyl group, a pyrazolidinyl group, an imidazolidinyl group, a piperidinyl group, a piperazinyl group, a morpolinyl group, a di(C₆-C₅₀ aryl)amino group, a di(C₆-C₅₀ aryl)aminophenyl group, a tri(C₆-C₅₀ aryl)silyl group and derivatives thereof.

8. The organic light emitting compound of claim 1, wherein R₁ and R₂ are each independently a methyl group, a methoxy group, a phenyl group, a tolyl group, a naphthyl group, an anthryl group, a pyrenyl group, a phenanthrenyl group, a fluorenyl group, an imidazolynyl group, an indolyl group, a quinolinyl group, a diphenylamino group, a 2,3-di-p-tolylaminophenyl group, a naphthylphenylamino group, a dinaphthylamino group, or a triphenylsilyl group and derivatives thereof.

9. The organic light emitting compound of claim 1, wherein the compound is represented by any one of Formulae 4 through 19:

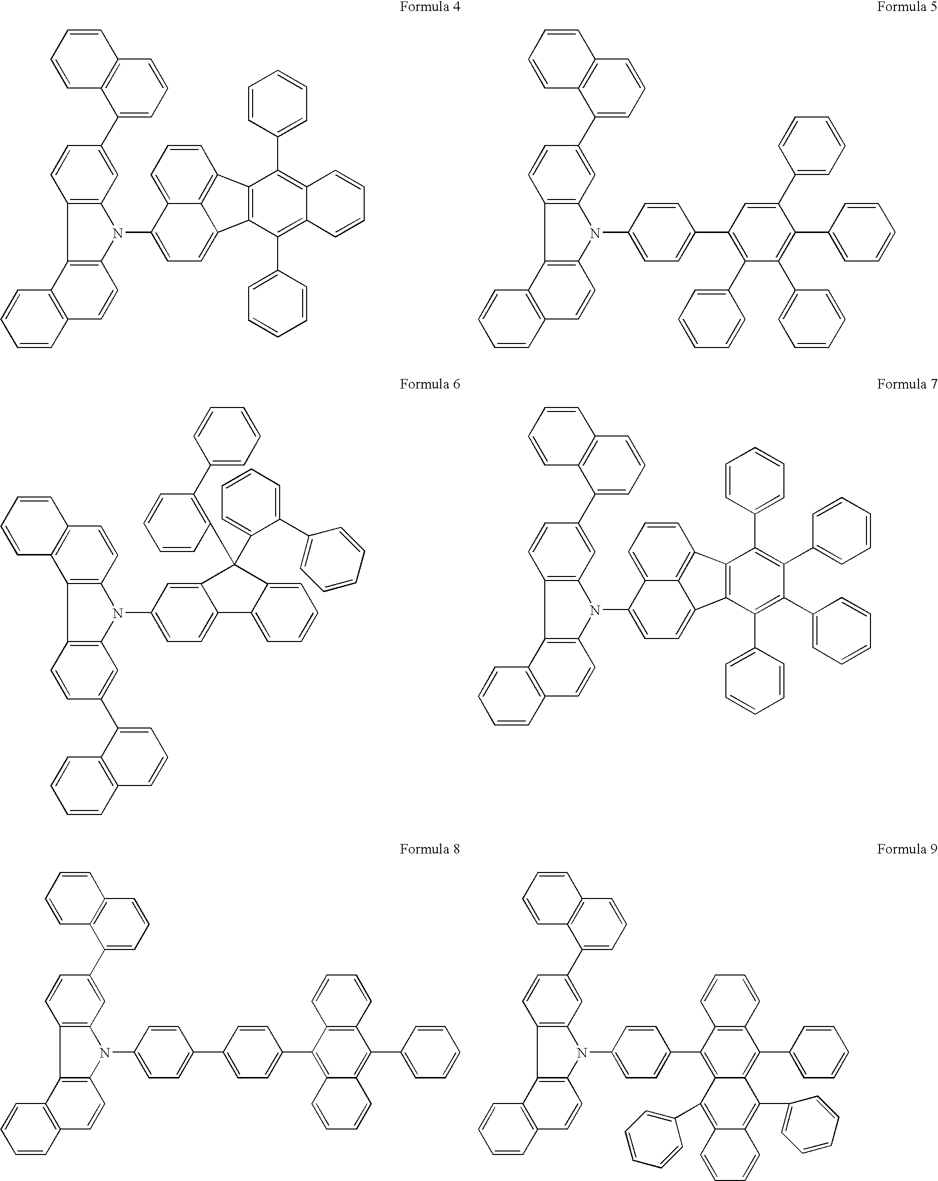

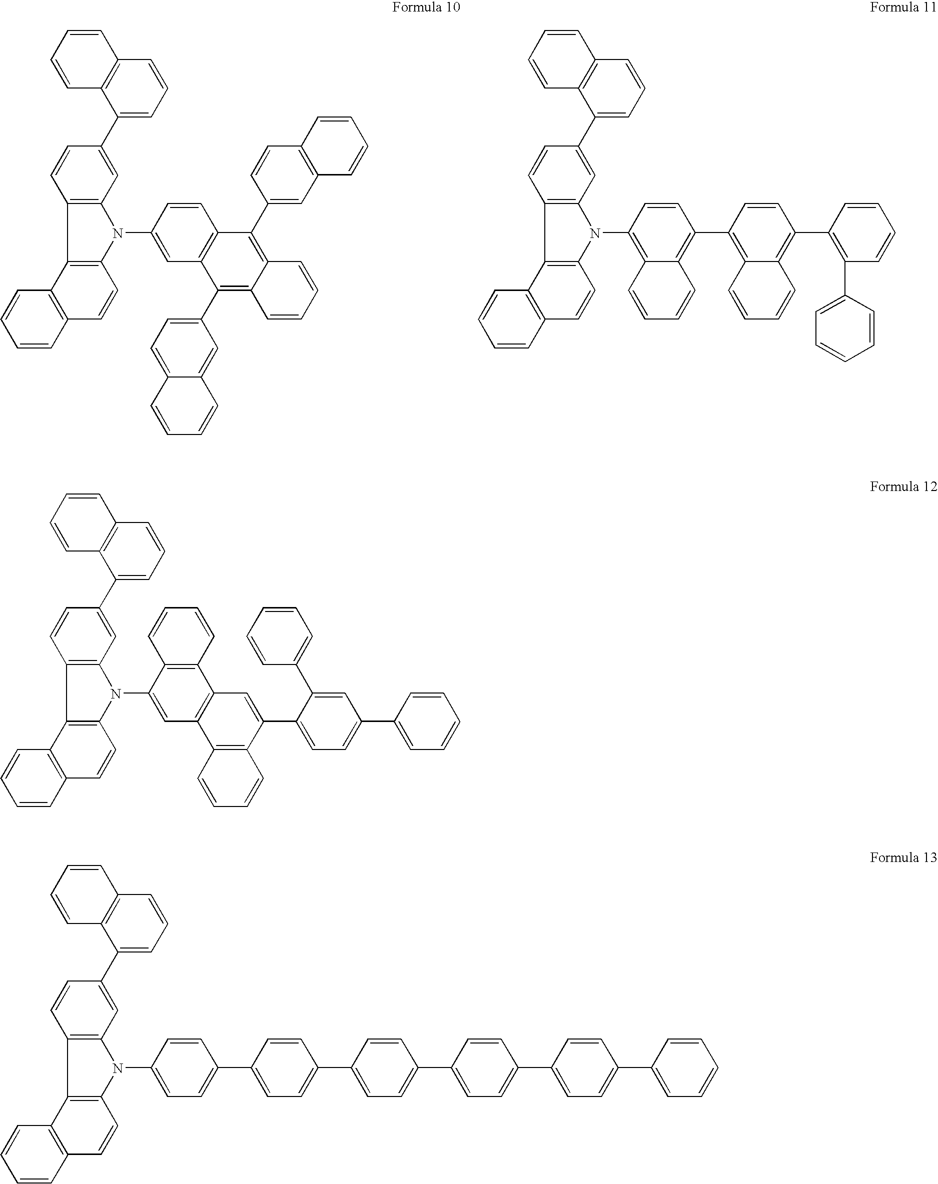

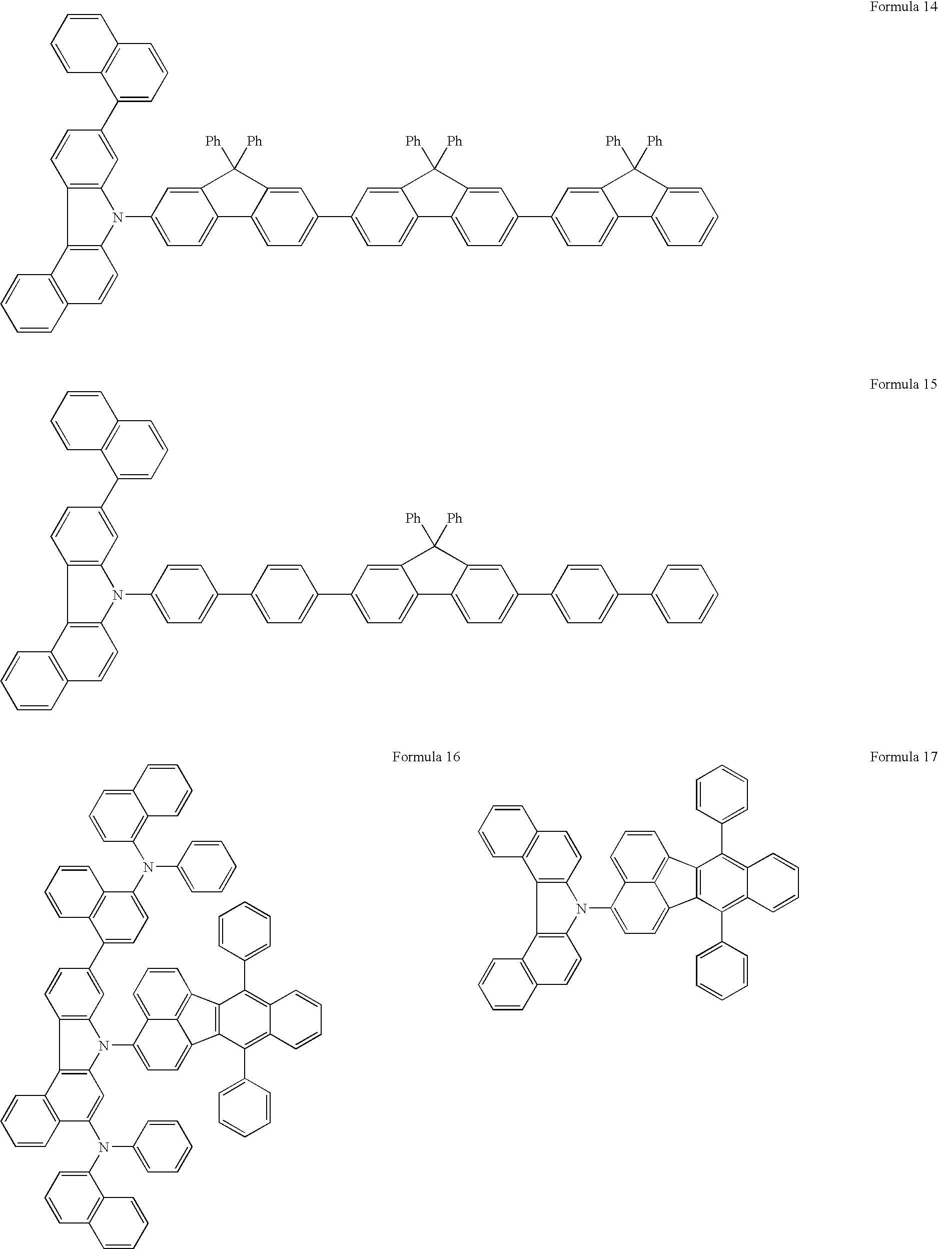

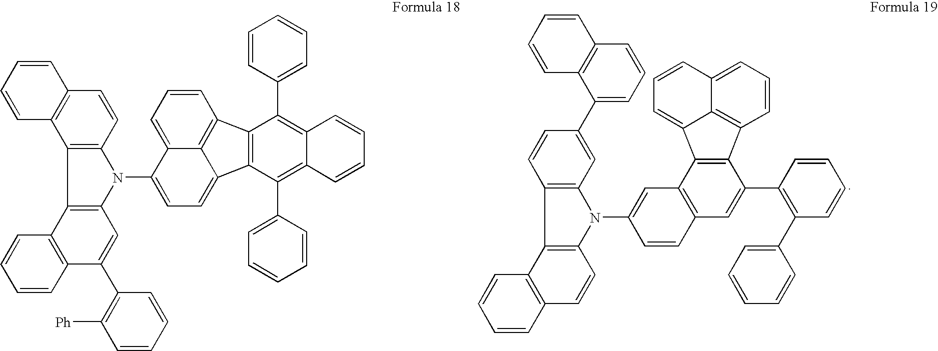

10. An organic light emitting device comprising: a first electrode; a second electrode; and an organic layer interposed between the first electrode and the second electrode, wherein the organic layer comprises an organic light emitting compound according to claim 1.

11. The organic light emitting device of claim 10, wherein the organic layer is an emission layer, a hole injection layer or a hole transport layer.

12. The organic light emitting device of claim 10, further comprising at least one layer selected from the group consisting of a hole injection layer, a hole transport layer, an electron blocking layer, a hole blocking layer, an electron transport layer and an electron injection layer, between the first electrode and the second electrode.

13. The organic light emitting device of claim 11, wherein the emission layer further comprises a red, green, blue or white phosphorescent or fluorescent dopant.

14. The organic light emitting device of claim 13, wherein the phosphorescent dopant is an organometallic compound comprising at least one atom selected from the group consisting of Ir, Pt, Os, Ti, Zr, Hf, Eu, Tb and Tm.

15. The organic light emitting device of claim 10, comprising a first electrode/hole injection layer/emission layer/electron transport layer/electron injection layer/second electrode structure, a first electrode/hole injection layer/hole transport layer/emission layer/electron transport layer/electron injection layer/second electrode structure or a first electrode/hole injection layer/hole transport layer/emission layer/hole blocking layer/electron transport layer/electron injection layer/second electrode structure.

16. A method of manufacturing an organic light emitting device comprising:
forming a first electrode;
forming an organic thin film comprising the organic light emitting compound according to claim 1 on the first electrode; and
forming a second electrode on the organic thin film.

17. The method of claim 16, wherein the organic thin film is formed using a wet spinning method or a heat transfer method.

18. The method of claim 17, wherein the wet spinning method is one selected from the group consisting of deposition, spin coating, inkjet printing and spray printing.
